(19) United States
(12) Patent Application Publication
(10) Pub. No.: US 2010/0022914 A1
(43) Pub. Date: Jan. 28, 2010

(54) Skin Biopsy Devices, Kits Containing Skin Biopsy Device, and Methods of Obtaining a Skin Biopsy

(76) Inventors: Mark Hart, Spokane, WA (US); William Werschler, Spokane, WA (US)

Correspondence Address: WELLS ST. JOHN P.S., 601 W. FIRST AVENUE, SUITE 1300, SPOKANE, WA, 99201, US

(21) Appl. No.: 12/519,148

(22) PCT Filed: Dec. 13, 2007
(86) PCT No.: PCT/US07/25488

§ 371 Date: Jun. 12, 2009

Related U.S. Application Data
(60) Provisional application No. 60/875,213, filed on Dec. 14, 2006.

(51) Int. Cl. A61B 10/02

(52) U.S. Cl. 600/567

(57) ABSTRACT

Skin-biopsy devices are provided that include a retractable coring assembly. Methods of acquiring a skin biopsy are provided that can include placing a lower portion of a skin-biopsy device proximate skin to be sampled, and extending at least a portion of a coring assembly from the device to acquire at least a portion of the skin to be sampled, and retracting the portion of the coring assembly to within the device, and discharging the portion of skin for analysis.

### RELATED PATENT DATA

The present disclosure relates to U.S. Provisional Patent Application Ser. No. 60/875,213 filed Dec. 14, 2006 and entitled “Skin Biopsy Device, Kit Containing A Skin Biopsy Device, and Methods Of Obtaining A Skin Biopsy”, the entirety of which is incorporated by reference herein and for all purposes.

### TECHNICAL FIELD

Skin biopsy devices, kits containing skin biopsy devices, and methods of obtaining skin biopsies.

### BACKGROUND OF THE DISCLOSURE

Skin biopsies may be utilized during diagnosis of various ailments, including cancer, rashes, fungal infections, etc. A significant portion of a physician's time may be dedicated to the taking of skin biopsies. Accordingly, it is desired to develop improved methods for obtaining skin biopsies which can be done more expeditiously than conventional methods. It is further desired to develop methods which are relatively simple procedures that can be done by persons other than physicians.

Another problem is that it is difficult for persons to have important diagnostic procedures, such as skin biopsies, performed in numerous locations due to the difficultly of locating physicians with appropriate training and tools. Such locations may include, for example, rural regions, regions occupied by military personnel, and regions of developing countries. It is desired to develop simple and expeditious methods for obtaining skin biopsies that may allow biopsy procedures to be readily performed even in such locations.

### SUMMARY OF THE DISCLOSURE

Skin-biopsy devices are provided that include a retractable coring assembly. Tissue coring apparatus are provided that can include a body extending from a first end to a second end, the body defining an opening and an interior wall extending to the opening at the first end. The apparatus can also include a plurality of tines configured to be received within the opening of the body, with the tines being biased toward the interior wall of the body.

Methods of acquiring a skin biopsy are provided that can include placing a lower portion of a skin-biopsy device proximate skin to be sampled, and extending at least a portion of a coring assembly from the device to acquire at least a portion of the skin to be sampled. Methods can also include retracting the portion of the coring assembly to within the device, and discharging the portion of skin for analysis.

Methods of acquiring a tissue core sample are provided that can include extending a plurality of coring tines from a tissue coring apparatus, both outwardly from an opening of the body of the apparatus, and laterally from an exterior of the body. The methods can also include retracting the plurality of coring tines, the retracting grappling tissue to be sampled with the plurality of tines.

### BRIEF DESCRIPTION OF THE DRAWINGS

Embodiments of the disclosure are described below with reference to the following accompanying drawings.

FIG. 1A illustrates operation of a skin biopsy tool embodiment of the disclosure.

FIG. 1B illustrates operation of another skin biopsy tool embodiment of the disclosure.

FIGS. 2 and 3 are a top view and a cross-sectional view of a region of skin having a lesion associated therewith. The view of FIG. 3 is along the line 3-3 of FIG. 2.

FIGS. 4-8 illustrate an embodiment for obtaining a skin biopsy.

FIGS. 9 and 10 are a cross-sectional side view and a top view, respectively, of a wound plugged after removal of a skin biopsy. FIG. 9 is along the line 9-9 of FIG. 10.

FIGS. 11 and 12 are a cross-sectional side view and a top view, respectively, of a coagulation foam embodiment.

FIGS. 13-19 illustrate an embodiment for obtaining a skin biopsy.

FIGS. 20A-20I are views of a skin-biopsy device according an embodiment of the disclosure.

FIG. 21 is an exploded view of the biopsy device of FIGS. 20A-20I according to an embodiment of the disclosure.

FIGS. 22-27 are views of components of the skin-biopsy device of FIGS. 20A-20I according to an embodiment of the disclosure.

FIG. 28 is a view of a skin-biopsy kit according to an embodiment.

### DETAILED DESCRIPTION

Embodiments of the disclosure include tissue coring apparatuses and skin-biopsy devices such as tools for extracting skin biopsies. Embodiments of the tools may function similarly to click-type pens and applications of the tools may permit non-physicians to safely obtain skin biopsies.

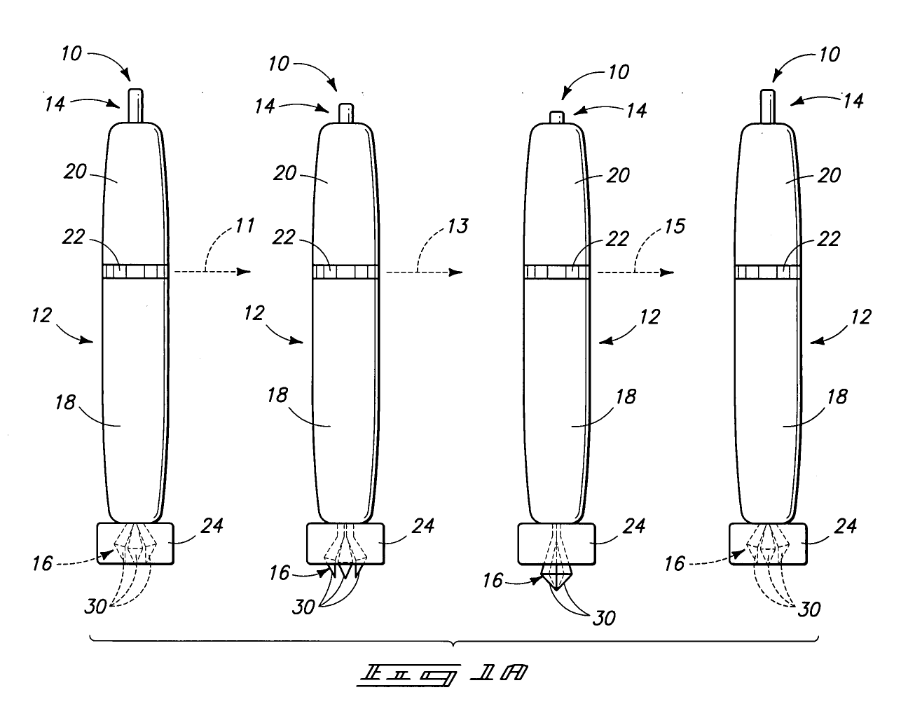

An example tool 10 is described in FIG. 1A. Tool 10 is shown in several orientations proceeding sequentially from the left-most side of the figure to the right-most side of the figure, with dashed arrows 11, 13 and 15 illustrating progression from one orientation to the next.

Referring initially to the left-most orientation, tool 10 comprises a body 12, a pushrod 14 extending into the body, and a coring mechanism 16 extending from the body (with the coring mechanism being retained in a lower portion of the body in the left-most orientation of FIG. 1A, and thus being shown in dashed-line view). The pushrod may be biased toward the shown fully-extended position by a spring (not shown) contained within the body 12.

In the shown embodiment, the body 12 comprises a pair of segments 18 and 20 joined to one another at a connection 22. In accordance with an example aspect, segment 18 can include the first end of tool 10 and segment 20 can include the second end of tool 10 defining body 12 extending from the first to the second end. Segments 18 and 20 may join through a threaded connection, or any other suitable connection. The segments 18 and 20 may be joined in a manner which can provide for tool 10 to be disassembled so that parts of the tool can be replaced to allow multiple uses of the tool, or the segments may be joined by a weld or other manner which precludes easy disassembly of the tool. According to example embodiments, segments 18 and 20 can respectively constitute upper and lower portions of tool 10. Coring mechanism 16 can extend from the lower portion of tool 10, for example.

Body 12 can define an opening and an interior wall extending to the opening at the first end for example. This opening and interior wall can be defined by segment 18. According to example implementations, mechanism 16 can be received within this opening of the body. The pushrod may be biased against body 12 to extend coring mechanism 16 from the lower portion of the body.

Body 12 can include a targeting segment 24 such as a tissue targeting assembly. The assembly can be associated with the first end of the body and can be within which the coring mechanism 16 is retained. According to example implementations, the targeting assembly can define at least a portion of a cylinder having a passageway there through, and the opening of the first end of body 12 can be aligned with the passageway of the cylinder. The first end of body 12 can further define a perimeter around the opening and the targeting assembly can extend around at least a portion of the perimeter defined by the first end. The targeting segment 24 may be transparent in some embodiments. The targeting segment 24 is configured to be placed over a region of skin where a biopsy is to be withdrawn, and may enable a user to accurately choose the skin region from which the biopsy will be removed.

In operation of acquiring a tissue core sample, a user can place targeting segment 24 of tool 10 on a region of skin where a biopsy is to be removed, and then depresses pushrod 14 such as a post. The post can be releaseably biased against at least a portion of body 12. The post can be biased against the body in a first position and released from bias upon attaining a second position. According to an example embodiment, in the first positions the tines of mechanism 16 can be fully retracted within the body and in the second position the tines are fully extended from the body. Depression of the pushrod against the bias and into body 12 from a first position to a second position can cause coring mechanism 16 to exit from the lower region of tool 10, as shown in the second orientation of the tool (specifically, the orientation following arrow 11). Mechanism 16 can include a coring assembly coupled to a plurality of coring tines such as three prongs (or petals) 30. The coring assembly can extend from the tines to the second end of body 12, for example. Individual ones of the tines can include a first end extending to a second end. The first end of the tines can define a cutting edge and the second end can be configured to couple to the coring assembly.

In other embodiments, the coring mechanism may comprise a different number of petals than three, or may comprise a different structure for obtaining a core sample of skin. The tines can be biased toward the interior wall of body 12 of tool 10, for example. The biasing can be accomplished by providing tines that are flexible, but resilient as well, returning to an original configuration when returned to a first position from a second position. Individual tines can include surfaces extending between the first and second ends of the tines. The surfaces can include an outer surface that can be oriented within the opening of the first end to oppose the interior wall of body 12. According to an example implementation, the individual tines can be oriented to align along an entirety of the interior wall of body 12 with the outer surfaces of the tines physically contacting at least a portion of the interior wall of body 12. The surfaces can also include an inner surface and the tines can be oriented within mechanism 16 to oppose the inner surface of another of the plurality of tines. The tines can be configured to extend from the opening of the first end of body 12 through the passageway of the cylinder defined by the targeting assembly.

In the shown embodiment, the three petals 30 are spaced from one another as the coring mechanism first exits body 12, and thus the coring mechanism is in an open configuration. The coring assembly of mechanism 16 can be configured to transition the individual tines between a first position and a second position, the first position being substantially within the body and biased between the interior wall of the body and an opposing tine. In the second position tines can be substantially outside the body and biased against a portion of the interior wall of the body. According to example implementations, the tines can be biased against at least a portion of the targeting assembly. The tines of mechanism 16 can be extended both outwardly from the opening of body 12 and laterally from an exterior of body 12, for example. The extending of mechanism 16 can engage the tissue to be sampled with a plurality of tines, and the engaging can include at least partially surrounding the tissue with the plurality of tines.

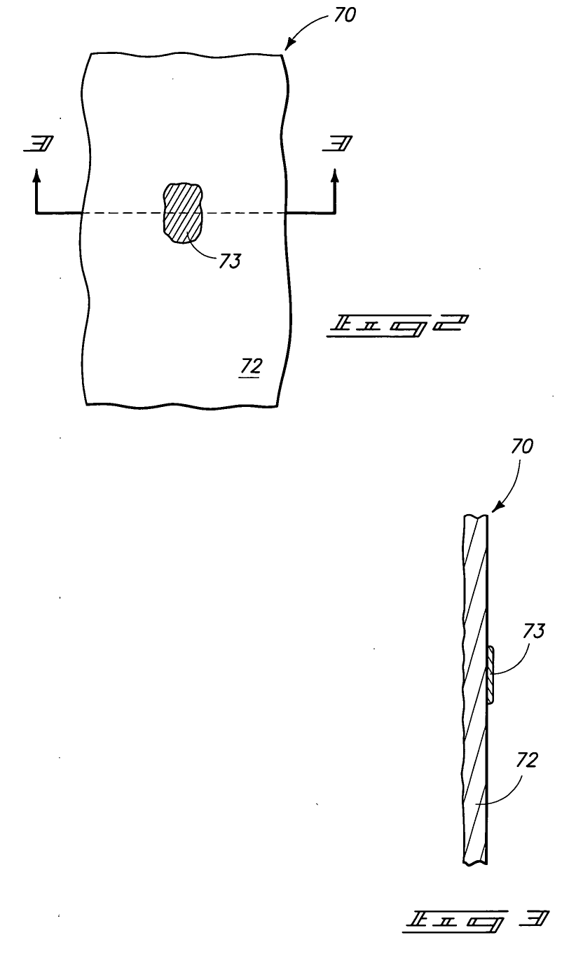

Further depression of pushrod 14 causes the tool to progress to the next orientation (specifically, the orientation following arrow 13). In such orientation, coring mechanism 16 exits further from body 12, and then the petals 30 come together to form a closed configuration. In operation, (described below with reference to FIGS. 4-6), such closed configuration can secure a sample of skin.

Release of pushrod 14 allows a biasing mechanism (not shown) within body 12 to retract coring mechanism 16 back into the body, and to extend the pushrod 14 back to the fully-extended position of the first orientation. Retracting of mechanism 16 can also include inserting pushrod 14 such as a post to the second position to release the bias against the post. Thus, release of pushrod 14 causes tool 10 to progress to a new orientation (specifically, the orientation following arrow 15) which is identical to the first orientation. According to an embodiment, the plurality of coring tines can be retracted and the retracting can grapple tissue to be sampled with the plurality of tines. Retracting can also include severing a portion of the tissue to be sampled that may be grappled from a remainder of the tissue. The biasing mechanism of the tool may comprise, for example, a spring and this biasing mechanism can be a portion of the coring assembly described herein.

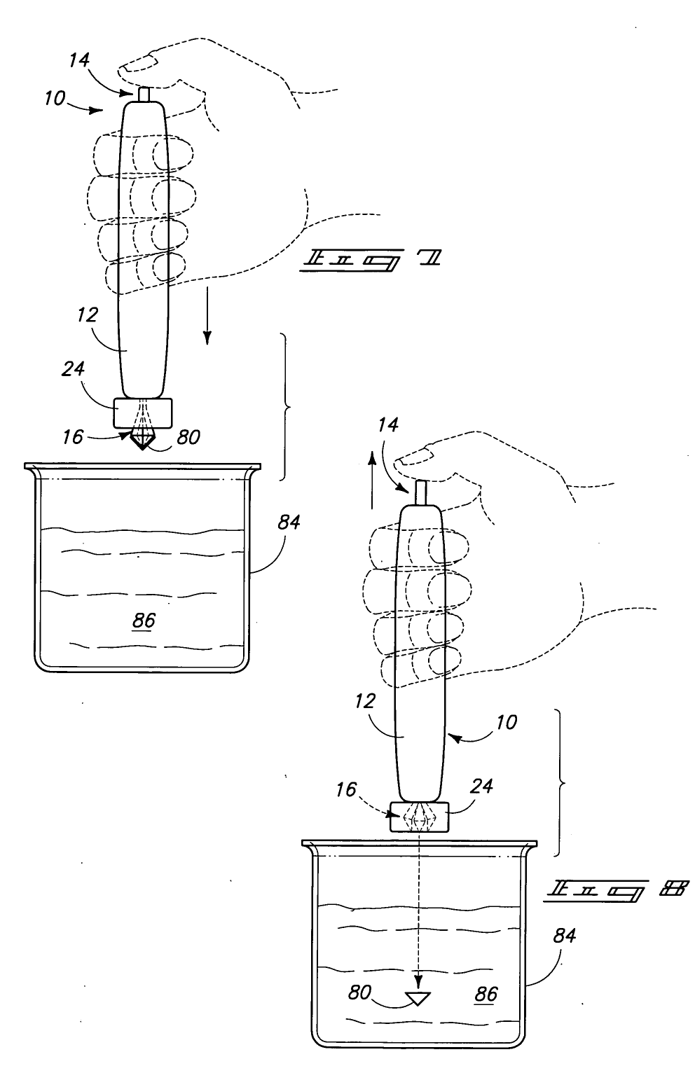

Retraction of coring mechanism 16 into the tool can also cause petals 30 to spread from one another, and thus may cause the coring mechanism to form the open configuration. In an operational sequence prior to being in this open configuration, a portion of the tissue can be retained with mechanism 16 to be discharged into a preservative fluid, for example. Such can cause release of a skin sample from the coring mechanism, as described below with reference to FIG. 8.

The embodiment of the skin biopsy tool described in FIG. 1A may be referred to as a one-click embodiment, in that each “click” of pushrod 14 causes the same action of coring mechanism 16. Specifically, when the pushrod is depressed the coring mechanism goes to the closed configuration, and when the pushrod is released the coring mechanism goes to the open configuration. Another embodiment of a skin biopsy tool is a two-click embodiment in which a first click leads to one orientation of the coring mechanism, and a second click leads to a different orientation of the coring mechanism. An example two-click embodiment is described with reference to a tool 50 in FIG. 1B. In referring to FIG. 1B, similar numbering will be used as is utilized above in referring to FIG. 1A, where appropriate. Tool 50 is shown in numerous orientations in FIG. 1B, with dashed arrows 51, 53, 55, 57 and 59 indicating progression from one orientation to another.

Referring initially to the left-most orientation of tool 50 in FIG. 1B, the tool comprises the body 12 and pushrod 14 discussed above in FIG. 1A. The body 12 comprises the pair of segments 18 and 20 joined to one another at connection 22, and comprises the targeting segment 24. The tool 50 also comprises the coring mechanism 16, having the petals 30. The coring mechanism 16 is in the open configuration in the left-most orientation of FIG. 1B.

Depression of pushrod 14 causes coring mechanism 16 to protrude from a bottom of tool 50, as shown in the orientation following dashed arrow 51.

Further depression of pushrod 14 causes coring mechanism 16 to protrude to its limit from the bottom of tool 50, and to change to the closed configuration, as shown in the orientation following dashed arrow 53.

Release of pushrod 14 causes retraction of coring mechanism 16. The retraction may bring the coring mechanism entirely into body 12 (as shown in the orientation following dashed arrow 55), or in other embodiments may bring the coring mechanism only partially into the body, or in yet other embodiments may bring the coring mechanism toward the body but not into the body. The coring mechanism remains in the closed configuration upon retraction. A subsequent push of pushrod 14 causes coring mechanism 16 to extend from body 12, as shown in the orientation following dashed arrow 57; and release of the pushrod causes coring mechanism 16 to retract back to the open configuration in which it started (as shown in the orientation following dashed arrow 59).

The tool 50 of FIG. 1B utilizes two clicks to return to the initial configuration. Specifically, the first click takes the tool through the configurations joined by arrows 51, 53 and 55, and the second click takes the tool through the configurations joined by arrows 57 and 59.

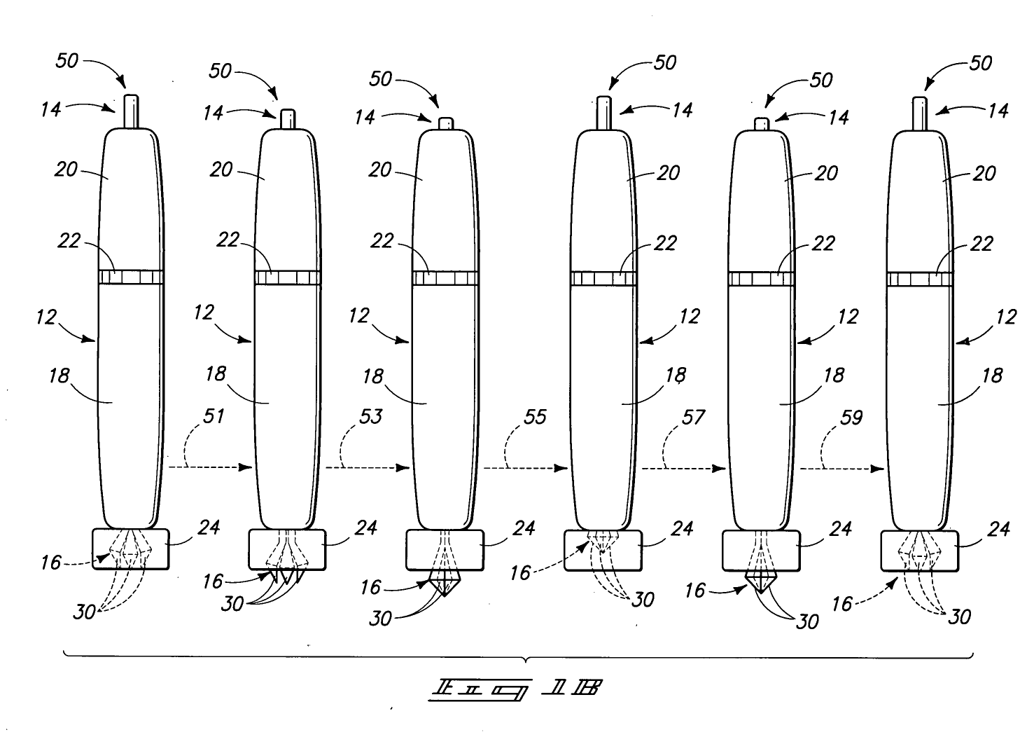

FIGS. 2 and 3 show a fragment 70 of skin 72 having a lesion 73 associated therewith. The lesion 73 may be anything desired to be biopsied, such as, for example, a dermatitis, (for instance, a rash), or a mole. Lesion 73 is shown along a surface of the skin, but it is to be understood that the lesion can also penetrate into one or more layers of the skin.

FIGS. 4-8 illustrate operation of the one-click tool 10 for taking a biopsy of lesion 73.

Referring to FIG. 4, targeting region 24 of tool 10 is aligned to lesion 73.

Referring to FIG. 5, pushrod 14 is depressed to cause coring mechanism 16 to extend into lesion 73 and close upon a sample 80 of the lesion. Tool 10 may be configured so that the coring mechanism extends to a depth of about three millimeters into the skin, and removes a cone-shaped core having a diameter of about three millimeters.

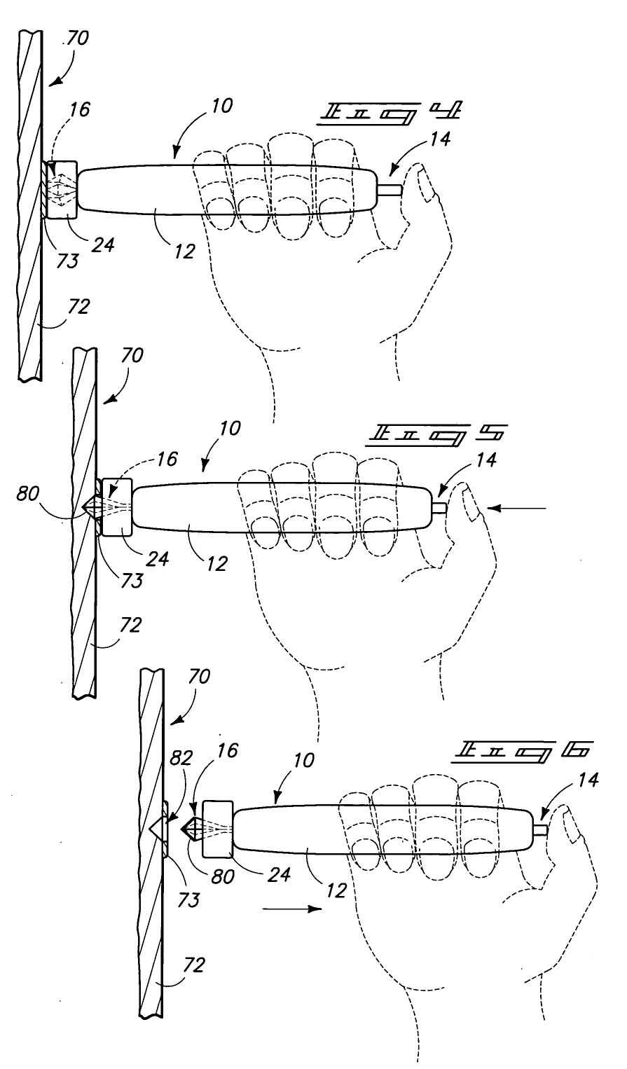

Referring to FIG. 6, tool 10 is withdrawn while maintaining depression of pushrod 14. Such takes the sample 80 from the patient, and leaves a wound 82 within the skin.

Referring to FIG. 7, tool 10 is held over a container 84 having a solution 86 retained therein. Solution 86 may be a preservative solution for preserving a skin sample for later analysis.

Referring to FIG. 8, pushrod 14 is released, which causes coring mechanism 16 to open and drop the skin sample 80 into solution 86. Container 84 may then be sealed, and provided to a lab where skin sample 80 may be analyzed to aid in medical diagnosis of the cause of lesion 73.

The wound 82 (FIG. 6) has a size corresponding to about the complement of the sample 80 withdrawn from such wound. Thus, if sample 80 is a cone having a height of about three millimeters and a diameter of about three millimeters, wound 82 may be cone-shaped with a depth of about three millimeters and a diameter of about three milliliters. The wound may be filled with a coagulation foam plug formed to appropriate size to fit snugly within the opening (in other words, formed to a size about the same as the size of the sample 80 removed from the skin).

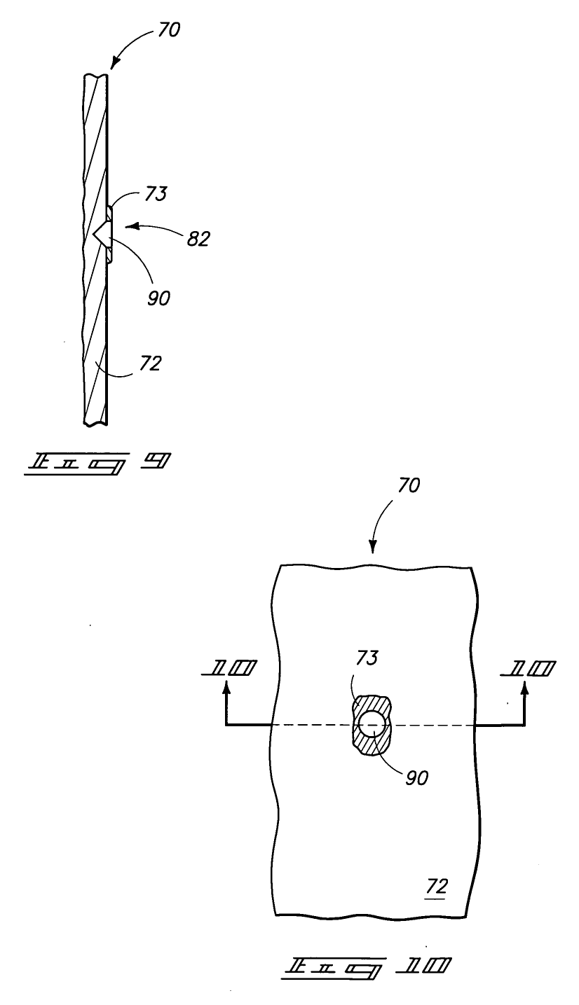

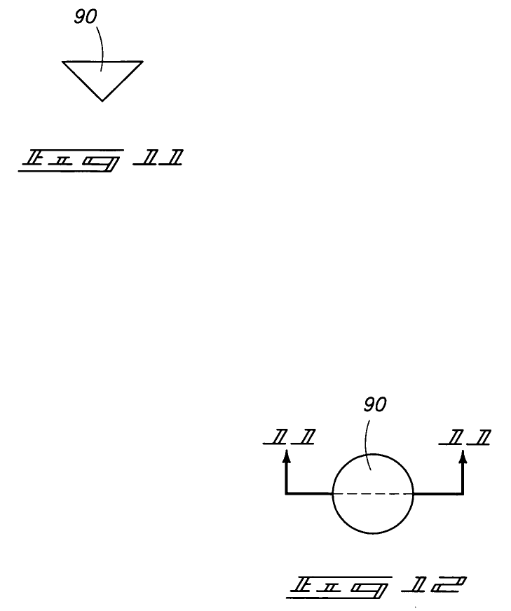

FIGS. 9 and 10 show skin fragment 70 at a stage subsequent to that of FIG. 6, and specifically after wound 82 has been filled with a coagulation foam plug 90. FIGS. 11 and 12 show a cone-shaped embodiment of coagulation foam plug 90.

The two-click tool 50 may be used for taking a biopsy of a lesion utilizing a process similar to that discussed above. FIGS. 13-19 illustrate operation of the two-click tool 50 for taking a biopsy of lesion 73.

Referring to FIG. 13, targeting region 24 of tool 50 is aligned with lesion 73.

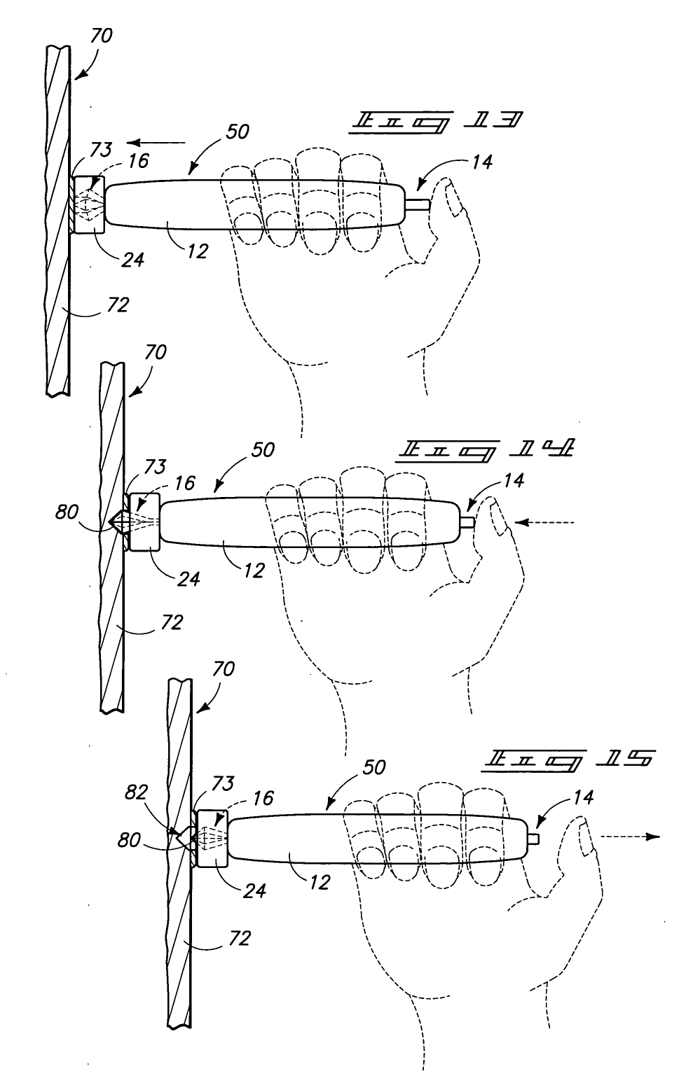

Referring to FIG. 14, pushrod 14 is depressed to cause the coring mechanism 16 to penetrate into lesion 73 and take a sample 80 of such lesion.

Referring to FIG. 15, pushrod 14 is released, which retracts coring mechanism 16. Such retraction withdraws sample 80 from skin 72. Mechanism 16 remains in a closed configuration after the first release of pushrod 14.

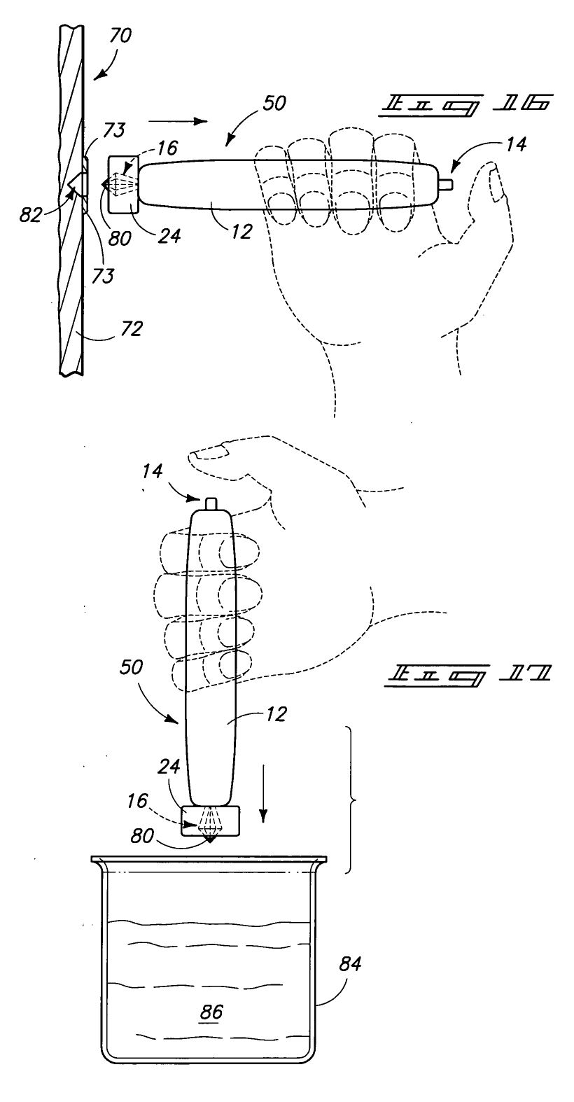

Referring to FIGS. 16 and 17, tool 50 is withdrawn from adjacent skin 72, and is then placed over container 84 having the solution 86 therein.

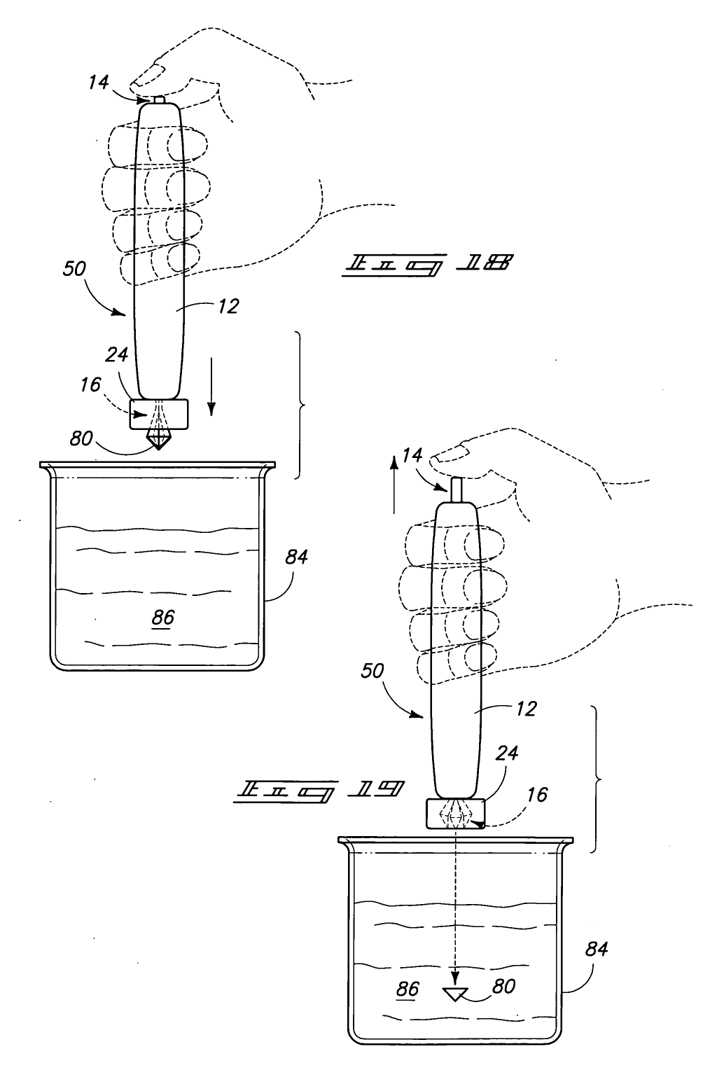

Referring to FIG. 18, pushrod 14 is depressed causing coring mechanism 16 to extend from body 12.

Referring to FIG. 19, pushrod 14 is released causing coring mechanism 16 to retract back toward body 12, and to open. The opening of coring mechanism 16 releases sample 80 into solution 86. The sample can then be provided to a lab for analysis.

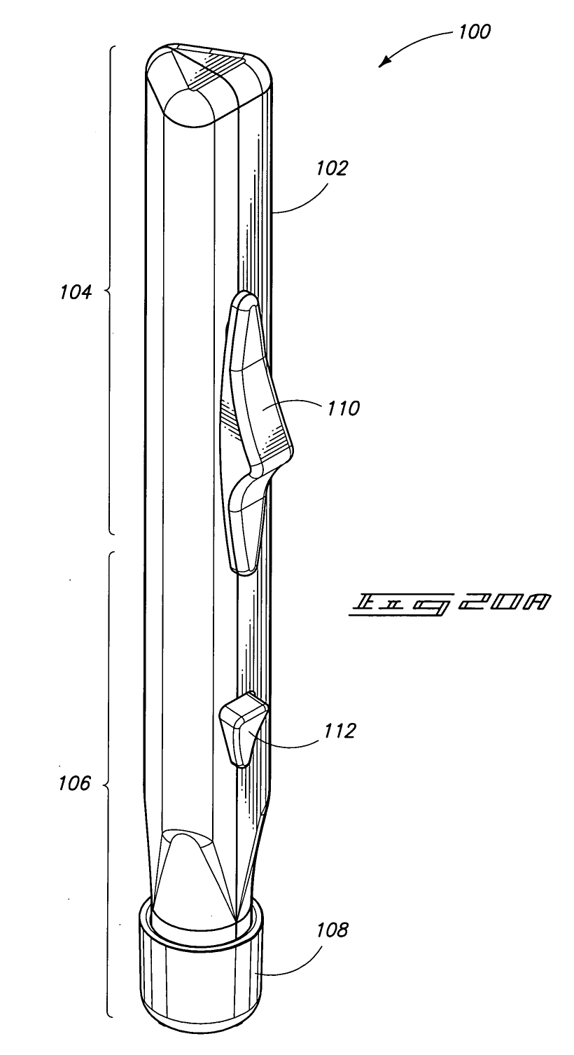

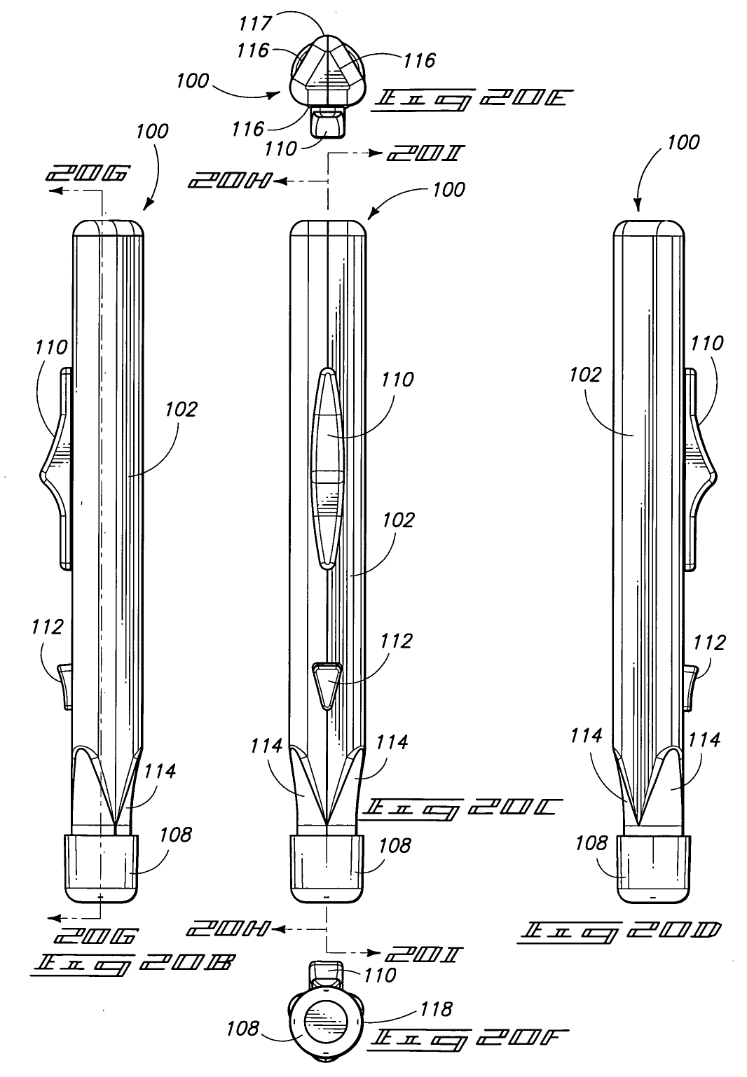

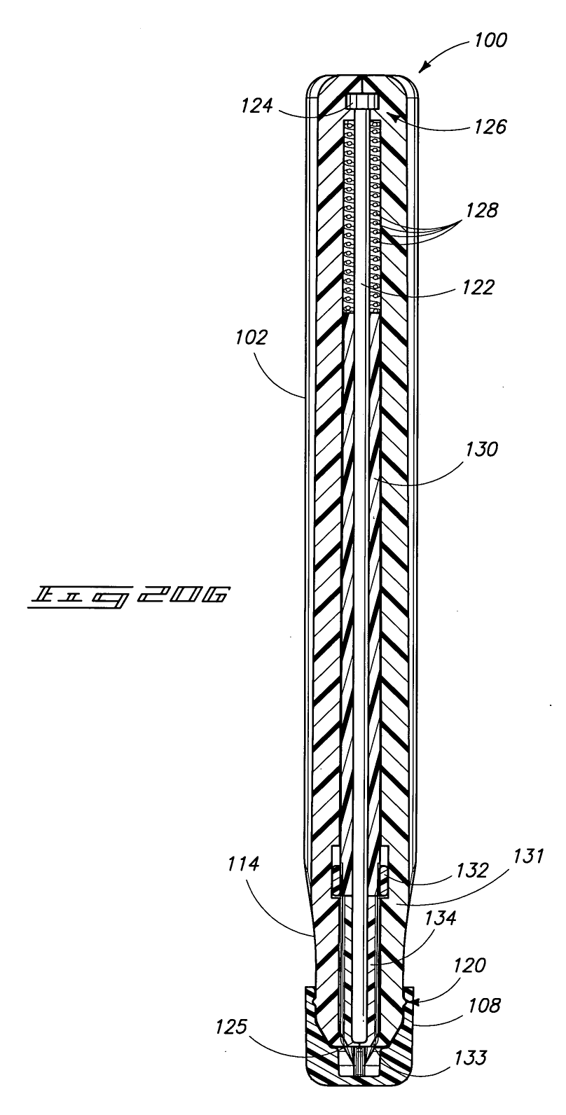

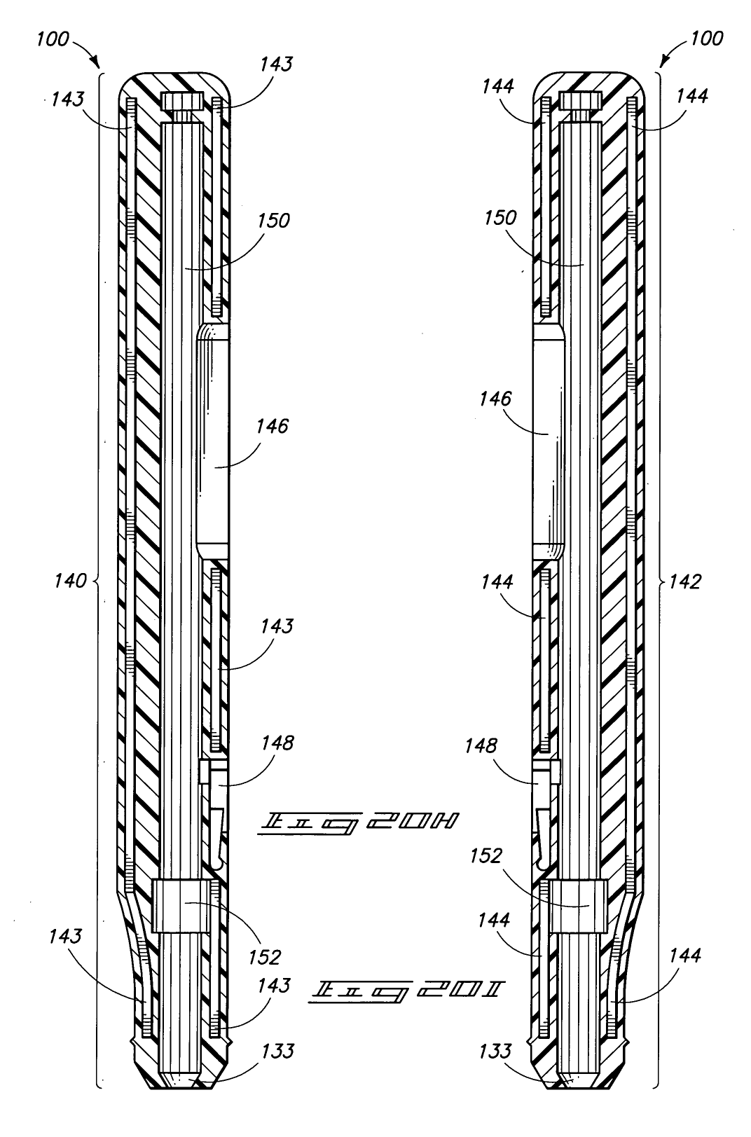

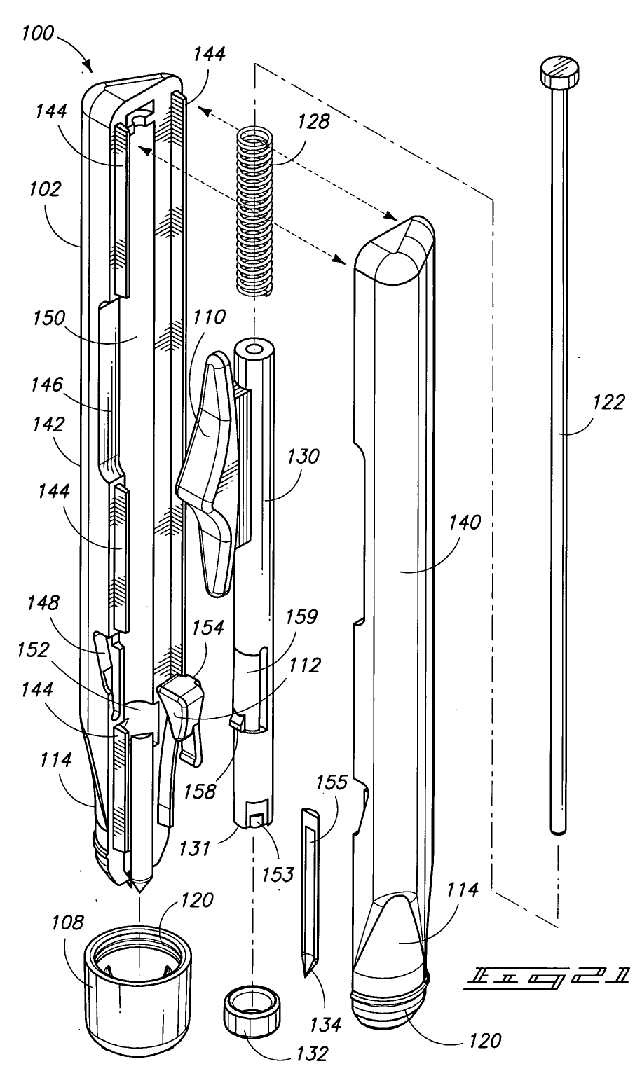

In accordance with another embodiment, a skin-biopsy device 100 is provided herein with reference to FIGS. 20-27. Referring to FIG. 20A, device 100 includes a body 102 that has respective upper and lower portions 104 and 106. Cap 108 can be removably coupled to a portion of body 102, for example. Extending laterally from body 102 is a hammer 110 and a trigger 112.

Referring to FIGS. 20B-20E, elevations of device 100 are depicted according to an embodiment. Referring to FIG. 20B, device 100 can include a facet portion 114 proximate lower portion 106. Facet portion 114 can be configured to ergonomically receive fingers of user during implementation of device 100. As an example, and referring to FIGS. 20B-20B, portions 114 can be received around a least a portion of the perimeter defined by portion 106 to provide a secure point to grasp device 100 during use.

In the depicted embodiment, hammer 110 and trigger 112 respectively can extend laterally from body 102 a sufficient distance to allow manipulation of these members by fingers during use, for example. According to one example, hammer 110 can extend laterally from body 102 a greater extent than trigger 112. This extension difference can allow a user to exert sufficient force against hammer 112 during cocking of device 100 described herein. Referring to FIG. 20C, hammer 110 and trigger 112 can be aligned along the long axis of device 100. Also aligned with hammer 112 and 110 along this long axis can be facets 114 which can be received with body 102 to compliment the opposing thumb and forefinger of a user, for example. Referring to FIGS. 20E and 20F respectively, top and bottom elevations of device 100 are depicted according to an embodiment. Body 102 of device 100 can be configured to define three sides 116 extending down its length with each side meeting a respective side at a curved corner 117. Cap 110 can have substantially curved sides 118 forming a portion of a cylinder closed at one end.

Referring to FIG. 20G a cross section of cross section of FIG. 20 B is depicted according to an embodiment. Body 102 is configured with a cavity to receive piston 130. Above piston 130, a biasing component 128 such as a spring is included and can be configured to bias piston 130 against lower portion 106 of body 102, for example. Piston 130 and component 128 can be configured to receive ejector rod 122 therein. Rod 122 can extend from upper portion 104 to lower portion 106 from ejector rod head 124 to ejector rod end 125, for example. Body 102 can be configured with rod head receiving portion 126 such as a recess to receive rod head 124. At least a portion of piston 130 can be received within blade sleeve 132, for example. Sleeve 132 can be received within another recess defined by body 102. At least a portion of sleeve 132 can receive at least a portion of tines 134 with tines 134 extending from one end 131 of piston 130 to an opening 133 defined by body 102.

Referring to FIGS. 20H and 20I, component portions 140 and 142 of device 100 are depicted according to an embodiment. These components can include complimentary coupling portions 143 and 144 respectively. Portions 143 and 144 can be configured to join via complimentary extensions and recessions, for example. According to example implementations, device 100 can be opened to reveal components of the coring assembly (e.g., rod 122, biasing component 128, piston 130, sleeve 132, and tines 134), for example. Any one or all of these or additional components may be maintained upon opening body 102 in this manner. Body 102 further defines hammer opening 146 extending into coring assembly receiving portion 150, as well as trigger opening 148, and/or sleeve receiving portion 152.

Referring to FIG. 21, an exploded view of device 100 is depicted according to an embodiment. Body 102 includes coupling portions 144 configured to removably couple complimentary portions of component 140. Body 102 further defines portion 150 configured to received piston 130, biasing component 128, tines 134, as well as rod 122. Body 102 further defines opening 146 extending to portion 150 and configured to receive an extension extending to hammer 110. Body 102 also defines opening 148 extending to portion 150 and configured to receive at least a portion of trigger 112. Sleeve 132 is configured to be received within receiving portion 152. Sleeve 132 can be configured to slidably engage a blade coupling extension 153 of piston 130 as it couples with channel recess 155 of tine 134. According to example implementations, extension 153 can compliment recess 155 to facilitate removable coupling of piston 130 and tines 134. Sleeve 132 can be aligned around end 131 of piston 130 to engage tines 134 with extensions 153.

According to an example implementation, trigger 112 can include extension 157 configured to engage complimentary extension 158 of piston 130. Extension 157 can be configured to maintain piston 130 in a cocked position against biasing component 128. Piston 130 can further include resilient portion 159 having extension 158 coupled thereto. Resilient portion 159 can be configured to receive compression force from a portion of trigger 112, such as extension 157, and such compression force can release piston 130 from its bias against component 128. Upon release, piston 130 slides towards opening 133 extending tines 134 therefrom.

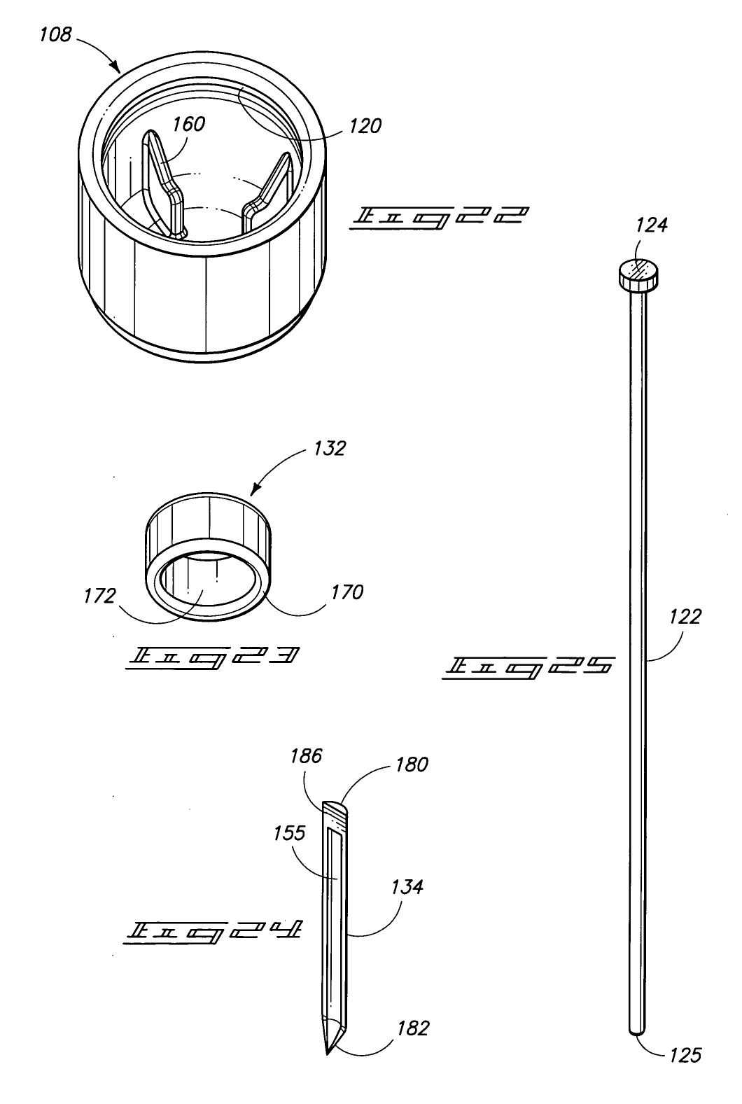

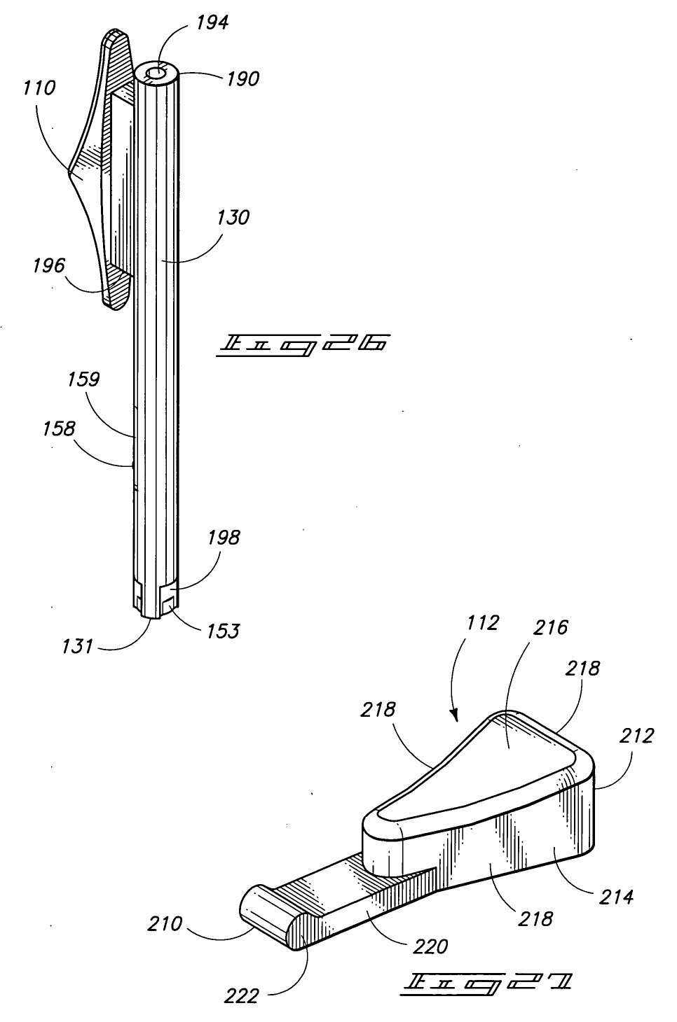

More detailed views of components of device 100 are depicted according to example embodiments in FIGS. 22-27. FIG. 22 depicts cap 108, and according to example embodiment, cap 108 includes tine receiving flanges 160 configured to keep tines aligned during non-use of device 100. FIG. 23 depicts sleeve 132 that defines an internal wall 172 configured to maintain the coupling of recess 155 and end portion 153. According to an example embodiment, edges 170 of sleeve 132 can be rounded. According to another embodiment these edges can be complimentary to those defined by guide receiving portion 152. FIG. 24 depicts an embodiment of tine 134 including channel 185 extending at least a portion of the length of tine 134 from first end 182 to second end 180. A portion 186 of second end 180 can be planar to compliment a portion of blade coupling portion of piston 132, for example. Second end 180 may also be blunt to facilitate coupling with piston 130. First end 182 can be sharpened to facilitate the severing of tissue. FIG. 25 depicts rod 122 extending from head 124 to end 125. End 125 can be aligned within device 100 to associate with tines 134. For example upon retrieving a sample, rod 122, particularly, end 125 can facilitate the ejection of the sample from tines 134.

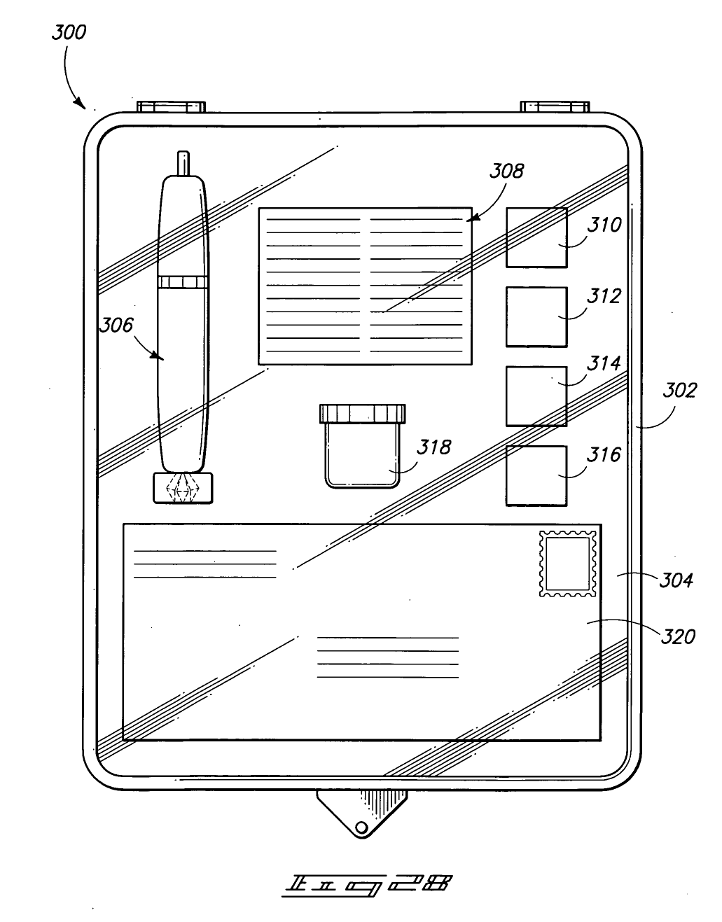

Referring to FIG. 26, an embodiment of piston 130 is shown. Piston 130 defines an opening 194 extending its length and configured to receive rod 122. Piston 130 further includes a member 196 extending to hammer 110. Member 196 can be configured to be received by opening 146 of body 102, for example. Piston 130 can also define a recess 198 configured to receive a portion of tines 134.

Referring to FIG. 27, an embodiment of trigger 112 is shown that includes a first end 210 extending to a second end 212. At least a portion of second end 212 can define a wall 214 extending to a surface 216 with surface 216 and wall 214 joining at curved edges 218 to form a triangular surface configured to ergonomically receive at least a single digit of a user. Extension 220 can extend from a portion of wall 214 to a pivot member 222. All or a portion of extension 220 can be formed of a resilient material. Pivot member 222 can be configured to pivot within a portion defined by body 102, for example.

According to an example implementation, upon removal of cap 108 from body 102, lower portion 106 can be placed proximate skin to be sampled. At least a portion of the coring assembly, particularly tines 134 can be extended from body 102 to acquire at least a portion of the skin to be sampled. This extension can be facilitated by first cocking piston 130 against the bias of component 128. According to an example implementation, force can be applied against hammer 110 upwards in the vertical direction to slide piston 130 within portion 150. To complete the cocking, extension 158 engages extension 154 of trigger 122. Upon manipulation of trigger 122, such as release, bias 128 forces piston 130 downwards in the vertical direction to extend tines 134 from body 102 and grapple and/or sever tissue as described above with respect to tools 10 and 50.

Upon extension of the coring assembly, it can be retracted to within body 102 of device 100. Upon retraction, rod 122 can discharge the portion of skin sampled for analysis. Skin sampled may also be discharged and stored prior to analysis as described above.

The biopsy tools discussed above can be provided in a kit so that the tools can readily be utilized by persons having little or no medical training. Such kits could be useful in, for example, rural, poor or military settings. An embodiment of a kit is shown in FIG. 28 as a kit 300. Such kit includes a carrying case 302. The carrying case is shown to comprise a transparent lid 304 through which various elements of the kit can be viewed.

The kit can comprise a biopsy tool 306 which may, for example, correspond to either tool 10, tool 50, and/or tool 100 discussed herein. The kit also includes an instruction sheet 308 providing instructions for using the various components of the kit. Additionally, the kit includes a package 310 containing anesthetic, a package 312 containing sterilization material, a package 314 containing coagulation foam, a package 316 containing a retaining material (such as a patch identified as a Band-Aid), a vial 318 containing preservative fluid, and an envelope 320 suitable for sending vial 318 to a lab for analysis of a skin sample contained therein.

In operation, the components of the kit may be utilized as follows. Initially, anesthetic (such as lidocaine) may be applied from package 310 to the area where a biopsy is to be taken. Subsequently the area may be sterilized utilizing a sterilization patch from package 312 (such as an alcohol wipe). Biopsy tool 306 may then be utilized to take a skin sample, and such sample may be transferred to vial 318 and sealed therein. The wound from which the skin sample is removed may be plugged with coagulation foam from package 314, and such foam may be held in place with retaining material from package 316. The vial may be placed within envelope 320 and sent to a lab for analysis.

Some components of the above-described kit may be optional, and some of the procedures may be done in an order other than that described.

After use, biopsy tool 306 may be disposed of if the tool is intended as a one-use tool, may be sterilized for reuse, and/or may have some parts (such as the coring mechanism) replaced with new, sterile parts.

What is claimed is:

1. A skin-biopsy device comprising a retractable coring assembly.

2. (canceled)

3. The skin-biopsy device of claim 1 further comprising a pushrod extending from the upper portion, the pushrod being mechanically coupled to the coring assembly.

4. (canceled)

5. (canceled)

6. The skin-biopsy device of claim 1 wherein the coring assembly comprises a plurality of tines, individual ones of the tines having sharpened edges.

7. The skin-biopsy device of claim 6 wherein the individual ones of the tines have a first pointed end extending to a second blunt end, the blunt end configured to mechanically couple another portion of the coring assembly and the pointed end configured to pierce skin tissue.

8. The skin-biopsy device of claim 7 wherein the individual ones of the tines further comprises opposing inner and outer surfaces extending between the first and second ends, the inner surface defining a channel extending at least partially between the first and second ends of the individual ones of the tines.

9. The skin-biopsy device of claim 1 wherein the device comprises a body having an upper portion and a lower portion, the retractable coring assembly being configured to extend from the lower portion and wherein the coring assembly comprises a piston coupled to a plurality of tines, the piston biased away from the upper portion of the body.

10. The skin-biopsy device of claim 9 further comprising a rod extending at least a portion of the length of the body, the rod having a first end associated with the tines and a second end coupled to the upper portion of the body.

11. (canceled)

12. The skin-biopsy device of claim 9 wherein the body has a side extending between the upper and lower portions, the body defining a first opening within the side, and wherein the piston further comprises a member extending from the piston and received by the first opening.

13. The skin-biopsy device of claim 9 wherein the body has a side extending between the upper and lower portions, the body defining a first opening within the side, and wherein a trigger member is received within the opening and operatively associated with the piston.

14. A tissue coring apparatus comprising:
a body extending from a first end to a second end, the body defining an opening and an interior wall extending to the opening at the first end; and
a plurality of tines configured to be received within the opening of the body, the tines being biased toward the interior wall of the body.

15. The tissue coring apparatus of claim 14 further comprising a tissue targeting assembly associated with the first end of the body.

16. The tissue coring apparatus of claim 15 wherein the first end further defines a perimeter around the opening and the targeting assembly extends around at least a portion of the perimeter.

17. The tissue coring apparatus of claim 16 wherein the targeting assembly defines at least a portion of a cylinder having a passageway therethrough, and the opening of the first end is aligned with the passageway of the cylinder.

18. The tissue coring apparatus of claim 17 wherein the tines are configured to extend from the opening of the first end of the body through the passageway of the cylinder of the targeting assembly.

19. (canceled)

20. (canceled)

21. (canceled)

22. (canceled)

23. (canceled)

24. A method of acquiring a skin biopsy comprising:
placing a lower portion of a skin-biopsy device proximate skin to be sampled;
extending at least a portion of a coring assembly from the device to acquire at least a portion of the skin to be sampled;
retracting the portion of the coring assembly to within the device; and
discharging the portion of skin for analysis.

25. (canceled)

26. (canceled)

27. (canceled)

28. The method of acquiring a skin biopsy of claim 24 wherein the placing the lower portion of the skin-biopsy device further comprises aligning a targeting assembly over the portion of skin to be sampled, the targeting assembly being associated with the lower portion of the device.

29. The method of acquiring a skin biopsy of claim 28 wherein the extending comprises extending at least a portion of the coring assembly through the targeting assembly to contact the portion of the skin to be sampled.

30. The method of acquiring a skin biopsy of claim 24 further comprising:
prior to extending the portion of the coring assembly, biasing a piston against an upper portion of the device with a trigger member; and
wherein the extending comprises releasing the trigger member to extend the portion of the coring assembly from the device.

31. The method of acquiring a skin biopsy of claim 30 wherein the retracting the portion of the coring assembly further comprises extending a rod member from within the piston through the coring assembly to forcibly discharge the portion of the skin.

32-41. (canceled)
